(19) United States
(12) Patent Application Publication
(10) Pub. No.: US 2009/0259135 A1
(43) Pub. Date: Oct. 15, 2009

(54) APPARATUS AND METHOD FOR CREATING MULTIPLE POLARITY INDICATING OUTPUTS FROM TWO POLARIZED PIEZOELECTRIC FILM SENSORS

(75) Inventor: Peter Stasz, Moundsview, MN (US)

Correspondence Address: SCHWEGMAN, LUNDBERG & WOESSNER, P.A., P.O. BOX 2938, MINNEAPOLIS, MN, 55402, US

(73) Assignee: Dymedix Corporation, Shoreview, MN (US)

(21) Appl. No.: 12/421,099

(22) Filed: Apr. 9, 2009

Related U.S. Application Data
(60) Provisional application No. 61/123,781, filed on Apr. 11, 2008.

(51) Int. Cl. A61B 5/08

(52) U.S. Cl. 600/534

(57) ABSTRACT

An apparatus or method can be configured to receive first information indicative of respiratory effort of a subject from a first piezoelectric film sensor and second information indicative of respiratory effort of the subject from a second piezoelectric film sensor, and to process the received first and second information to produce an electronic signal output indicative of respiratory effort of the subject, the processing including averaging the received first information using a first differential amplifier and signal integrator with resistive reset to reduce differential noise and to attenuate common-mode noise, and averaging the received second information using a second differential amplifier and signal integrator with resistive reset to reduce differential noise and to attenuate common-mode noise.

### CLAIM OF PRIORITY

This patent application claims the benefit of priority to U.S. Provisional Patent Application Ser. No. 61/123,781, filed on Apr. 11, 2008, which application is herein incorporated by reference in its entirety.

### BACKGROUND

I. Field of the Invention

This invention relates generally to an electronic signal processing circuit for adapting two polarized piezoelectric film sensor based respiratory effort belts to a conventional polysomnograph (PSG) machine of the type commonly used in sleep laboratory applications, and more particularly to an adapter that receives one polarized polyvinylidene fluoride (PVDF) film sensor signal from a chest movement respiratory effort belt and second polarized PVDF film sensor signal from an abdominal movement respiratory effort belt.

The invention creates multiple polarity indicating signal outputs from two polarized piezoelectric film sensors that are integrated into the respiratory effort belts. Therefore, a sleep disorder-diagnosing professional is being presented with a plurality of respiratory waveforms indicating whether a sleeping patient is breathing normally during sleep or indicating whether a patient is suffering from a sleep disorder.

II. Discussion of the Prior Art

In addressing sleep related problems, such as sleep apnea, insomnia and other physiologic events or conditions occurring during sleep, various hospitals and clinics have established laboratories sometimes referred to as “Sleep Laboratories” (sleep labs). At these sleep labs, using instrumentation, a patient's sleep patterns may be monitored and recorded for later analysis so that a proper diagnosis may be made and a therapy prescribed. Varieties of sensors have been devised for providing recordable signals related to respiratory patterns during sleep. These sensors commonly are mechanical to electrical transducers that produce an electrical signal related to body movement.

For example in the Pennock U.S. Pat. No. 4,960,118, a method and apparatus for accurately measuring respiratory flow, while the subject breathes, without using a mouthpiece, face mask or any device about the head is described. The rate of change of the circumference of the rib cage (chest) and the rate of change of the circumference of the abdomen are measured using an extensible belt with series strips of polarized piezoelectric film sensors mounted thereon. The stress on the film produces an electric output proportional to the rate of application of stress when the sensor connected to a proper electronic amplifier. Calibration is performed by measuring the circumferential changes while the subject performs an isovolume maneuver for several breaths or while the subject breathes through a pneumotachometer and mouthpiece at a variable rate for several breaths. Calibration, as described in the Pennock U.S. Pat. No. 4,960,118, is cumbersome and puts and unnecessary strain and discomfort on the patient.

In another example in the Watson U.S. Pat. No. 4,373,534 entitled “Method and Apparatus for Calibrating Respiration Monitoring System”, extensive calibration is also required. The method described in the '534 patent is known in the sleep medicine industry as “Respiratory Inductance Plethysmography (RIP)”. It is a well-known matter of fact within the sleep industry that RIP technology requires extensive calibration and RIP belt measurements are actually prone to polarity reversals during the sleep tests and thus have a negative impact on the official scoring of the individual sleep patients results.

In yet another example in the Watson U.S. Pat. No. 4,834,109 entitled “Single Position Non-Invasive Calibration Technique”, RIP technology calibration is at the core of the invention. It is clear to persons skilled in the art that calibration is a key factor in the application and success of RIP technology.

### SUMMARY

The present inventor has recognized, among other things, that there is a need to provide an apparatus and method that does not require the patient to go through an extensive calibration procedure as described in the Pennock U.S. Pat. No. 4,960,118, Watson U.S. Pat. No. 4,373,534 and Watson U.S. Pat. No. 4,834,109.

An apparatus and method that uses two independent polarized piezoelectric film sensors in order to provide a rigid phase and polarity relationship between respiratory effort movement (inhaling and exhaling) to final graphical indication of the individually processed polarized piezoelectric film sensor signals on the PSG machine display is needed.

Furthermore, there is also a need to provide an apparatus and method capable of using two independent polarized piezoelectric film sensors in order to provide a rigid phase relationship between respiratory effort movement (inhaling and exhaling) to final graphical indication of the summed polarized piezoelectric film sensor signals on the PSG machine display.

The polarized piezoelectric film sensor based respiratory effort belts of the present invention can contain PVDF film sensors, which act like a pre-charged polarized capacitor that provides changes in capacitive reactance (impedance) during breathing and non-breathing events. In comparison, Respiratory Inductance Plethysmography (RIP) belts consists of an array of specially arranged wires that must be locally excited by a low current, high frequency external electrical oscillator circuit. Although small, this external electrical oscillator unit is subject to wear and tear, signal loss, and frequent replacement, at significant cost, not to mention replacement of the expensive RIP belts.

To successfully market, these new types of polarized piezoelectric film sensors along with electronic sensor signal adapters, it is desirable that they not require extensive calibration and do not provide false respiratory effort results during sleep tests while still being able to be used with existing polysomnograph machines already in place in sleep laboratories.

Certain embodiments of the present invention provide an adaptor for interfacing two polarized piezoelectric film sensors to a PSG machine. The adapter comprises two independent sets of differential amplifier and integrator circuits with resistive reset having a pair of input terminals that are adapted to be coupled to the polarized piezoelectric film sensor and output terminal. The differential amplifier and signal integrator with resistive reset are configured to provide a predetermined gain factor by which the polarized piezoelectric film sensor output signal is amplified, to provide input signal averaging over time to reduce unwanted differential noise, and to significantly attenuate common-mode noise. Each of the outputs of the two differential amplifier and signal integrator with resistive reset circuits is fed to a set of signal output attenuators and to a summing node and to two stage inverting signal integrators with resistive reset.

By utilizing a differential input amplifier with a predetermined gain factor and by appropriately conditioning the amplified polarized piezoelectric film sensor output signal, the resulting three different filters can be readily matched to existing PSG electronic head boxes already on hand in most sleep laboratories.

A non-obvious aspect of the invention is that the adapter itself requires no calibration. There are no adjustable components as part of this assembly that might require tuning during a calibration procedure.

In certain examples, the integrator with resistive reset circuit can be crucial for the application when the two polarized piezoelectric film sensors are part of a respiratory effort belt system because PVDF film senses mechanical motion/stresses well into the GHz range. Because the normal human adult at rest respiration rate is relatively slow (12 to 20 breaths per minute), irregular in frequency and amplitude and of distorted sinusoidal form, the raw polarized piezoelectric film sensor signals must be averaged over time in order to condition the signal for graphical presentation in the PSG machine. Sensor signal averaging over time removes undesired patient motion signals and other environmentally induced noise signal artifacts.

The two Belt Sensors comprising the present invention are applied to a patient during sleep study recordings. The Belt Sensor provides a small voltage signal in relationship to a mechanical change due to breathing. When the chest belt sensor is being stretched, then the patient is inhaling. The signal is provided to the user's external sleep recording device. Trained medical professionals examine the recording to provide an assessment of breathing by chest and abdominal cavity movements during sleep.

Polarized lead wires are provided to interface between the belt Sensors and the user recorder. One wire may be outfitted with a red marking and is designated positive. The other wire may be outfitted with a black marking and is designated negative. The PVDF film generates a direct current (DC) voltage, much like a battery, when subjected to a mechanical stress such as stretching during inhaling. Inhaling means that the PVDF film is being stretched. Exhaling results in that the PVDF film is becoming relaxed. Lead Wires are provided to interface between the Sensor and user recorder. The positive designated PVDF film surface electrode becomes negatively charged when exhaling. The positive designated PVDF film surface electrode becomes positively charged when inhaling. The negative designated PVDF film surface electrode becomes negatively charged when inhaling. The negative designated PVDF film surface electrode becomes positively charged when exhaling. The PSG display is indicated by an upward deflection when inhaling.

When a negative voltage/charge is presented to the PSG input reference terminal, an upward deflection on the PSG display indicates a respiratory exhalation effort. When a positive voltage/charge is presented to the PSG input reference terminal, a downward deflection on the PSG display indicates a respiratory inhalation effort.

In order to maintain the polarity processing properties and to minimize potentially long phase delays as part of the invention, all electronic signal-processing paths are DC coupled. Persons skilled in the art will recognize the DC coupling when reviewing the disclosed schematic diagram of the invention by the absence of capacitors in the forward signal paths in any of the electronic building blocks.

Further areas of applicability of the present invention will become apparent from the description provided herein. It should be understood that the description and specific examples are intended for purposes of illustration only and are not intended to limit the scope of the present disclosure.

In an example, multiple polarity indicating outputs can be created using two polarized piezoelectric film sensors. Typically, the two polarized piezoelectric film sensors are constructed of polyvinylidene (PVDF) film and are part of a set of respiratory effort sensing belts. The apparatus contains a chest signal-processing channel and an abdominal signal-processing channel. The input section of the chest signal-processing channel consists of a differential amplifier and integrator with resistive reset coupled to receive the polarized piezoelectric film signal from the PVDF film transducer that makes up the sensing apparatus in the chest effort-sensing belt. The input section of the abdominal signal-processing channel consists of a differential amplifier and integrator with resistive reset coupled to receive the polarized piezoelectric film sensor signal from the PVDF film transducer that makes up the sensing apparatus in the abdominal effort-sensing belt. Both chest and abdominal differential amplifiers and integrators with resistive reset provide load impedance, voltage gain and signal averaging over time while rejecting differential and common mode noise. The output of the chest differential amplifier and integrator with resistive reset connects to the input of a third order Butterworth low-pass filter for further signal shaping and conditioning. The output of the abdominal differential amplifier and integrator with resistive reset connects to the input of a third order Butterworth low-pass filter for further signal shaping and conditioning. The outputs of the chest signal third order Butterworth low pass filter and the output of the abdomen signal third order Butterworth low pass filter are added in a summing node of a sum channel two-stage signal integrator with resistive reset. The shaped output of the sum signal two-stage integrator with resistive reset passes through the sum signal output attenuator for conditioning so that the sleep therapy professional recognizes the resulting summed chest and abdomen movement on the polysomnograph machine (PSG) more commonly. In addition, each shaped output of the chest and abdomen third order Butterworth low pass filters pass through separate chest and abdominal signal attenuators for conditioning so that the specific PSG machine displays the resulting waveforms optimally.

In Example 1, an apparatus includes an electronic signal processing circuit configured to receive first information indicative of respiratory effort of a subject from a first piezoelectric film sensor and second information indicative of respiratory effort of the subject from a second piezoelectric film sensor, wherein the electronic signal processing circuit is configured to process the received first and second information to produce an electronic signal output indicative of respiratory effort of the subject, the electronic signal processing circuit including a first differential amplifier and signal integrator with resistive reset configured to average the received first information to reduce differential noise and to attenuate common-mode noise, and the electronic signal processing circuit including a second differential amplifier and signal integrator with resistive reset configured to average the received second information to reduce differential noise and to attenuate common-mode noise.

In Example 2, the apparatus of Example 1 optionally includes a first respiratory effort belt including the first piezoelectric film sensor configured to sense movement indicative of respiratory effort of the subject, and a second respiratory effort belt including the second piezoelectric film sensor configured to sense movement indicative of respiratory effort of the subject.

In Example 3, the first respiratory belt of any one or more of Examples 1-2 optionally includes a chest movement respiratory effort belt and the second respiratory effort belt includes an abdominal movement respiratory effort belt.

In Example 4, the first and second piezoelectric film sensors of any one or more of Examples 1-3 optionally include independent polarized piezoelectric film sensors configured to provide a rigid phase and polarity relationship between respiratory effort movement.

In Example 5, the first and second differential amplifiers and signal integrators with resistive resets of any one or more of Example 1-4 are optionally configured to provide a predetermined gain factor by which the received first and second information is amplified, to provide signal averaging over time to reduce differential noise, and to attenuate common-mode noise.

In Example 6, the electronic signal processing circuit of any one or more of Examples 1-5 optionally includes a first third order Butterworth low pass filters configured to remove components from the received first information having a frequency above approximately 500 mHz, and a second third order Butterworth low pass filters configured to remove components from the received second information having a frequency above approximately 500 mHz.

In Example 7, the first and second differential amplifiers and signal integrators with resistive reset of Examples 1-6 are optionally configured to average the received first and second information only during the subject's respiratory response time.

In Example 8, the electronic signal output indicative of respiratory effort of the subject of any one or more of Examples 1-7 optionally includes a summation of the averaged received first and second information.

In Example 9, the electronic signal output indicative of respiratory effort of the subject of any one or more of Examples 1-8 optionally includes the averaged received first information, and separately, the averaged received second information.

In Example 10, the electronic signal output indicative of respiratory effort of the subject of any one or more of Examples 1-9 optionally includes, separately, the averaged received first information, the averaged received second information, and a summation of the averaged received first and second information.

In Example 11, a system includes a first respiratory effort belt including a first piezoelectric film sensor configured to sense a first movement indicative of respiratory effort of a subject, a second respiratory effort belt including a second piezoelectric film sensor configured to sense a second movement indicative of respiratory effort of the subject, an electronic signal processing circuit, coupled to the first and second piezoelectric film sensors, the electronic signal processing circuit configured to receive first and second information from the first and second piezoelectric film sensors and to process the received first and second information to produce an electronic signal output indicative of respiratory effort of the subject, wherein the electronic signal processing circuit includes a first differential amplifier and signal integrator with resistive reset configured to average the received first information to reduce differential noise and to attenuate common-mode noise, and wherein the electronic signal processing circuit includes a second differential amplifier and signal integrator with resistive reset configured to average the received second information to reduce differential noise and to attenuate common-mode noise in the second, wherein the electronic signal output indicative of respiratory effort of the subject includes the averaged received first information, the averaged received second information, and a summation of the averaged received first information and the averaged received second information, and wherein the system includes a polysomnograph machine, coupled to the electronic signal processing circuit, the polysomnograph machine configured to receive the averaged received first information, the averaged received second information, and the summation of the averaged received first information and the averaged received second information from the electronic signal processing circuit and to provide the averaged received first information, the averaged received second information, and the summation of the averaged received first information and the averaged received second information to a user.

In Example 12, a method includes receiving first information indicative of respiratory effort of a subject from a first piezoelectric film sensor and second information indicative of respiratory effort of the subject from a second piezoelectric film sensor, processing the received first and second information to produce an electronic signal output indicative of respiratory effort of the subject, the processing including averaging the received first information using a first differential amplifier and signal integrator with resistive reset to reduce differential noise and to attenuate common-mode noise, and the processing including averaging the received second information using a second differential amplifier and signal integrator with resistive reset to reduce differential noise and to attenuate common-mode noise.

In Example 13, the receiving the first and second information of Example 12 optionally includes receiving first and second information from the first and second piezoelectric film sensors coupled to respective first and second respiratory effort belts.

In Example 14, the first respiratory effort belt of any one or more of Examples 12-13 optionally includes a chest movement respiratory effort belt and the second respiratory effort belt of any one or more of Examples 12-13 optionally includes an abdominal movement respiratory effort belt.

In Example 15, the first and second piezoelectric film sensors of any one or more of Examples 12-14 optionally includes independent polarized piezoelectric film sensors, wherein the receiving the first and second information of any one or more of Examples 12-14 optionally includes receiving rigid phase and polarity relationship information between respiratory effort movement from the independent polarized piezoelectric film sensors.

In Example 16, the processing of any one or more of Examples 12-15 optionally includes amplifying the received first and second information by a predetermined gain factor using the first and second differential amplifiers and signal integrators with resistive reset, to provide signal averaging over time to reduce differential noise, and to attenuate common-mode noise.

In Example 17, the processing of any one or more of Examples 12-16 optionally includes removing components from the received first and second information having a frequency above approximately 500 mHz using respective first and second third order Butterworth low pass filters.

In Example 18, the averaging the received first and second information of any one or more of Examples 12-17 optionally includes averaging only during the subject's respiratory response time.

In Example 19, the producing the electronic signal output indicative of respiratory effort of the subject of any one or more of Examples 12-18 optionally includes producing a summation of the averaged received first and second information.

In Example 20, the producing the electronic signal output indicative of respiratory effort of the subject of any one or more of Examples 12-19 optionally includes producing the averaged received first information, and separately, the averaged received second information.

### DESCRIPTION OF THE DRAWINGS

The drawings described herein are for illustration purposes only and are not intended to limit the scope of the present disclosure in any way.

The forgoing features, objects and advantages of the invention will become apparent to those skilled in the art from the following detailed description of a preferred embodiment, especially when considered in conjunction with the accompanying drawings in which like the numerals in the several views refer to the corresponding parts:

FIG. 1 is a general block diagram of the adapter module comprising a preferred embodiment of the present invention;

FIG. 2 is a more detailed block diagram of the adapter module comprising a preferred embodiment of the present invention; and

FIG. 3 is a schematic diagram of the adapter module comprising a preferred embodiment showing a detailed implementation thereof.

### DESCRIPTION OF THE PREFERRED EMBODIMENT

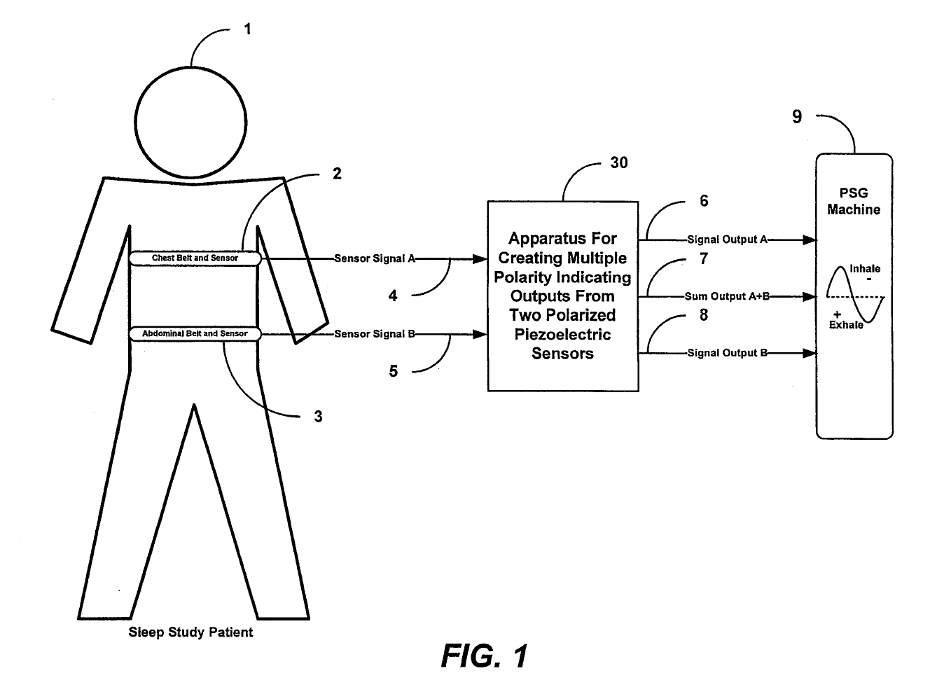

The present invention can be readily understood from FIGS. 1 through 3 and the following description.

The following description is merely exemplary in nature and is not intended to limit the present disclosure, application, or uses. It should be understood that throughout the drawings, corresponding reference numerals indicate like or corresponding parts and features.

A typical overall use and configuration of the adapter module is shown with the aid of FIG. 1. Referring to FIG. 1, there is indicated generally by numeral 1 a typical sleep laboratory patient who has been outfitted with one chest belt 2 and one abdominal belt 3 to measure respiratory effort. Each belt includes an integral polarized piezoelectric film sensor as may be found in the Stasz invention “Respiratory Sensing Effort Belt Using Piezo Film”, Ser. No. 11/743,839, filed May 3, 2007, the teachings of which are hereby incorporated by reference. A pair of chest belt sensor output wire leads 4 and a pair of abdominal belt sensor output wire leads 5 connect the sleep laboratory patient 1 to the adapter apparatus for creating multiple polarity indicating outputs from two polarized piezoelectric film sensors 30.

This specific exemplary of the embodiment shows three polarity indicating output wire pairs 6, 7 and 8 connecting the apparatus for creating multiple polarity indicating outputs from two polarized piezoelectric film sensors to a conventional, commercially-available PSG machine 9, such as a

Model Sandman available from Covidien of Kanata, ON Canada
Model Alice available from Respironics Inc of Murrysville, Pa.
Model Connex available from Natus Medical of Oakville, ON Canada
Model Harmonie-S available from Stellate of Montreal, QC Canada
Model Polysmith available from Nihon Kohden America of Foothill Ranch, Calif.
Model Comet available from Astro-Med, Inc of West Warwick, R.I.
Model Embletta available from Embla of Broomfield, Colo.
Model E Series available from Compumedics of Charlotte, N.C.
Model 20B available from CleveMed of Cleveland, Ohio
Model Somnostar available from Cardinal Health of Yorba Linda, Calif.
Model Easy II available from Cadwell Laboratories, Inc of Kennewick, Wash.
Model Pursuit Sleep2 available from Braebon of Ogdensburg, N.Y.
Model SleepScan available from Natus Medical of Mundelein, Ill.
All trademarks are property of their respective owners! This list is only exemplary in nature and does not claim to be comprehensive or complete.

In a typical sleep laboratory application, signal output A 6 is configured to produce the chest movement showing inhalation as an upward deflection of respiratory effort and showing exhalation as a downward deflection of respiratory effort on the PSG machine 9 display.

Furthermore, sum signal output A+B 7 is configured to produce the sum of the phase and amplitude for the chest movement sensor signal and the abdominal movement sensor signal as an upward deflection of respiratory effort during inhalation and showing exhalation as a downward deflection of respiratory effort on the PSG machine 9 display.

Furthermore, signal output B 8 is configured to produce the abdominal movement signal from the piezoelectric film sensor associated with the abdominal belt 3 showing inhalation as an upward deflection of respiratory effort and showing exhalation as a downward deflection of respiratory effort on the PSG machine 9 display.

It is by international convention and by requirement of the American Association of Sleep Medicine (AASM) that a patient's inhalation produces an upward deflection and exhalation produces a downward deflection on the PSG machine 9 display.

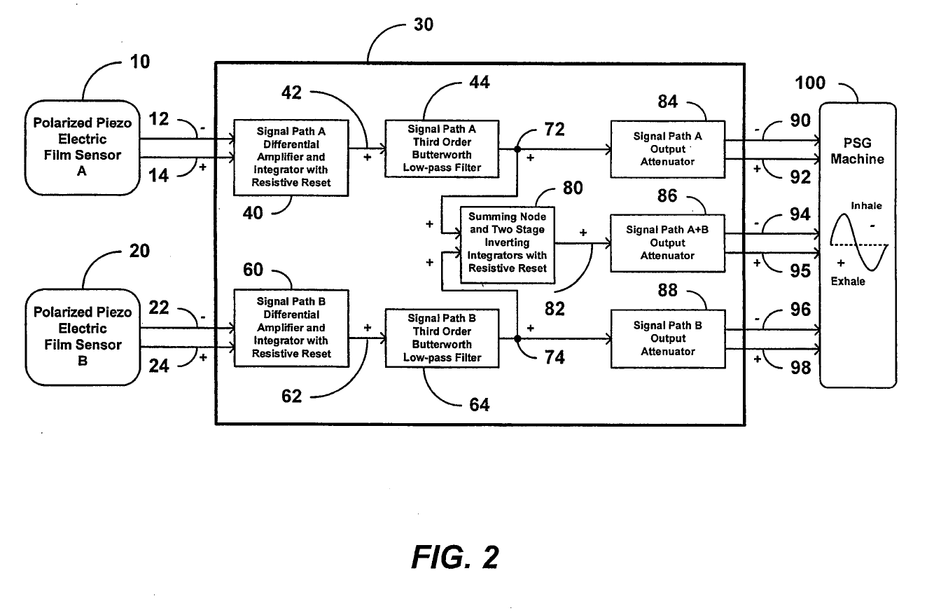

Referring next to FIG. 2, there is indicated generally by numeral 30 the functional components comprising the adapter module of the present invention.

The polarized piezoelectric film sensor A 10 in general and more specifically, the chest belt sensor, is preferably constructed in accordance with the teachings of the aforereferenced patent application Ser. No. 11/743,839 of Peter Stasz and entitled “Respiratory Sensing Belt Using Piezo Film”. The sensor 10 is adapted to be placed on a subject's chest so that inspiratory and expiratory chest cavity movements create mechanical stress on the sensor as the belt 2 stretches and contracts.

The polarized piezoelectric film sensor A 10 connects to the signal path A differential amplifier and integrator with resistive reset 40 via a pair of input wire leads 12-14.

Wire 12 of the input wire pair is indicated to represent the positive terminal of the polarized piezoelectric film sensor that goes positive when patient is inhaling.

Wire 14 of the input wire pair is indicated to represent the negative terminal of the polarized piezoelectric film sensor that goes positive when patient is inhaling. The differential amplifier and integrator with resistive reset 40 of the signal path A comprises a differential type amplifier which functions to increase the common-mode rejection of the adapter system so as to make it less susceptible to 60 Hz noise present in the environment as well as to motion artifacts. The signal integrator with resistive reset serves to slowly average the incoming signal over time so that the differential amplifier only amplifies signals that are within the response time of interest, i.e., the patient's respiratory response time. The averaging signal integrator may operate with a fixed time constant of about 62.5 ms. This value has been selected and found to be working optimally during operation and performance regarding respiratory effort.

Without limitation, the differential amplifier and integrator with resistive reset 40 may have a gain in the range of from 2 to 10 with about 6.2 being quite adequate.

The output signal 42 from the differential input amplifier and integrator with resistive reset 40 is applied to a third order Butterworth low pass filter 44. The input of the third order Butterworth filter 44 is connected to the output terminal 42 of the differential input amplifier 40.

It should be understood by those skilled in the art that the type of filter response is neither limited to a third order filter nor is it limited to a Butterworth response. Other filter responses may also be used.

Typically, but not limited to, the cut-off frequency for the third order Butterworth low pass filter 44 may be about 500 mHz.

The output 72 of the signal path A third order Butterworth low-pass filter module 44 connects to the input of the signal path A output attenuator module 84.

The output attenuator for signal path A 84 attenuates the signal coming from the signal path A third order Butterworth low-pass filter 44 in order to reduce the signal path A amplitude to a level that is compliant with the requirements of the input specifications of the input jack of the PSG machine 100 by way of lines 90 and 92 respectively.

It should be clear to those skilled in the art that the entire signal path A starting from the polarized piezoelectric film sensor 10 and ending at the PSG machine 100 is DC coupled, thus ensuring that the relationship of polarized piezoelectric film sensor polarity and indication of respiration effort between inhalation and exhalation on the PSG machine is purposely maintained.

The polarized piezoelectric film sensor B 20 in general and more specifically, the chest belt sensor, is also preferably constructed in accordance with the teachings of the aforereferenced Peter Stasz application entitled “Respiratory Sensing Belt Using Piezo Film”. The sensor B 20 is adapted to be placed on a subject's abdomen so that inspiratory and expiratory belly movements create mechanical stress on the sensor.

The polarized piezoelectric film sensor B 10 connects to the signal path B differential amplifier and integrator with reset 60 via a pair of input wire leads 22-24.

Wire 22 of the input wire pair is indicated to represent the positive terminal of the polarized piezoelectric film sensor that goes positive when patient is inhaling.

Wire 24 of the input wire pair is indicated to represent the negative terminal of the polarized piezoelectric film sensor that goes positive when patient is inhaling. The differential amplifier and integrator with resistive reset 60 of the signal path A comprises a differential type amplifier which functions to increase the common-mode rejection of the adapter system so as to make it less susceptible to 60 Hz noise present in the environment as well as to motion artifacts. The signal integrator with resistive reset functions to slowly average the incoming signal over time so that the differential amplifier only amplifies signals that are within the response time of interest, more specifically the patient's respiratory response time. The averaging signal integrator preferably operates with a fixed time constant of about 62.5 ms. This value has been selected and found to be working optimally during operation and performance regarding respiratory effort.

Without limitation, the differential amplifier and integrator with resistive reset 60 may have a gain in the range of from 2 to 10 with about 6.2 being quite adequate.

The output signal 62 from the differential input amplifier and integrator with resistive reset 60 is also applied to a third order Butterworth low pass filter 64. The input of the third order Butterworth filter 64 is connected to the output terminal 62 of the differential input amplifier and integrator with resistive reset 60.

It is to be understood by those skilled in the art that the type of filter response is neither limited to a third order filter nor is it limited to a Butterworth response. Other filter responses may be used.

Typically, the cut-off frequency for the third order Butterworth low pass filter 64 may be about 500 mHz.

The output 74 of the signal path B third order Butterworth low-pass filter 64 connects to the input of the signal path B output attenuator 88.

The output attenuator for signal path B 88 attenuates the signal coming from the signal path B third order Butterworth low-pass filter 64 in order to reduce the signal path B amplitude to a level that is compliant with the requirements of the input specifications of the input jack of the PSG machine 100 by way of lines 96 and 98 respectively.

It should be clear to those skilled in the art that the entire signal path B starting from the polarized piezoelectric film sensor 20 and ending at the PSG machine 100, is DC coupled. This ensures that the relationship of polarized piezoelectric sensor film polarity and indication of respiration effort between inhalation and exhalation on the PSG machine is purposely maintained.

In order to create polarity indicating signal path A (chest belt sensor) and signal path B (abdominal belt sensor) sum output for connection and presentation to the PSG machine 100 display, the output 72 of the signal path A third order Butterworth low-pass filter 44 and the output 74 of the signal path B third order Butterworth low-pass filter 64 are added in the summing node of a two-stage inverting integrators with resistive reset 80.

Summing node and two stage inverting integrators with resistive reset 80 slowly average the summed signal path A and signal path B (chest and abdomen) output signals 72 plus 74 over time so that the signals that are outside the integrating time constant are rejected. The averaging signal integrator is preferably but not necessarily operating with a fixed time constant of about 62.5 ms that has been selected and found to be working optimally during operation and performance regarding respiratory effort.

The output line 82 of the summing node and two stage-inverting integrators with resistive reset 80 connects to the input of the signal path A+B output attenuator 86.

The output attenuator for signal path A+B 86 attenuates the signal coming from the summing node and two stage inverting integrators with resistive reset 80 in order to reduce the signal path A+B amplitude to a level that is compliant with the requirements of the input specifications of the input jack of the PSG machine 100 by way of lines 94 and 95 respectively.

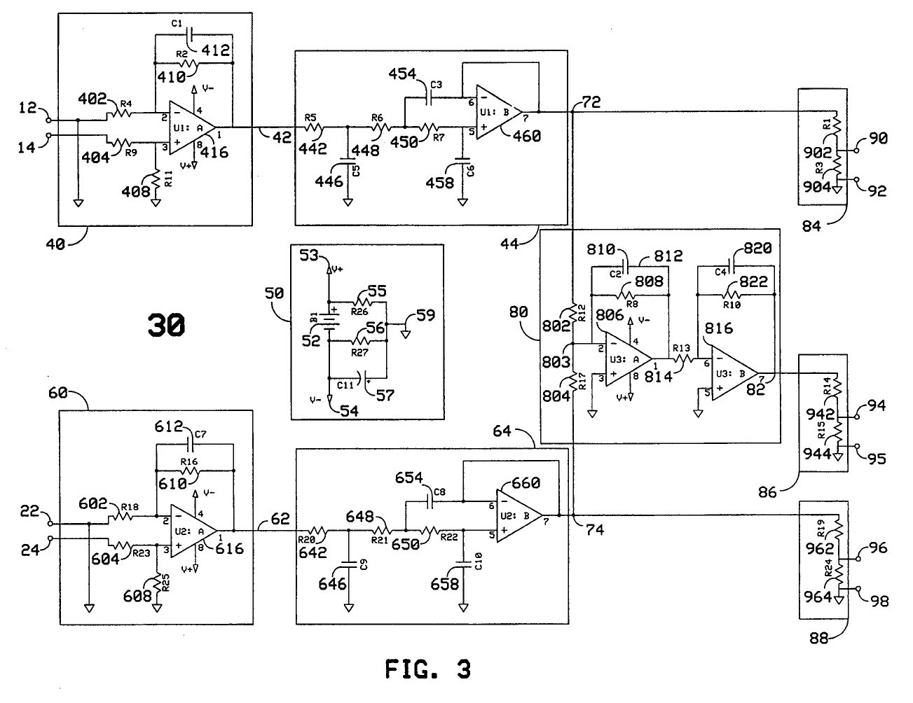

Having described the overall configuration of the adapter module with the aid of FIG. 2, a more detailed explanation of a specific implementation of the adapter will now be presented and, in that regard, reference is made to the schematic diagram of FIG. 3. FIG. 3 describes in detail the building blocks outlined in FIG. 2.

The adapter 30 of the present invention is integral with the cable used to couple the two polarized piezoelectric film (chest and abdominal) sensors 10 and 20 respectively to the polysomnograph machine. As such, it incorporates its own power supply and virtual ground generator 50 in the form of a single lithium battery 52 with its positive battery voltage terminal 53 identified as v+ and its negative battery voltage terminal 54 labeled v− The resistor 55 connects the positive battery voltage terminal to the virtual ground point 59. The resistor 56 connects the negative battery voltage terminal to the virtual ground point 59. Resistors 55 and 56 are equal in value in establishing virtual ground point 59. The polarized capacitor 57 connects in parallel with resistors 56 to form a low alternating current (AC) impedance return path from the negative battery terminal 54 to the virtual ground point 59.

The input terminal 12 to the differential amplifier and integrator with resistive reset 40 is coupled, via resistor 402 to the inverting input of operational amplifier 416, to the gain setting and integrator resetting resistor 410 and to the integrating capacitor 412. The input terminal 14 connects to the non-inverting input of differential operational amplifier and integrator with resistive reset 116, via resistor 404 and to the input load resistor 408.

The output from the differential input amplifier circuit 416 appears at junction 42 and connects to the signal path A third order Butterworth low-pass filter circuit 44.

Referring to filter circuit 44, the input appearing at junction 42 is applied, via series connected resistors 442, 448 and 450, to the non-inverting input of an operational amplifier 460 and those resistors, along with capacitors 446, 454 and 458 cooperate with the operational amplifier 460 to function as a low-pass filter. The output of the operational amplifier 460 is presented to node 72.

The values of the resistors 442, 448 and 450 and the capacitors 446, 454 and 458 may be set to establish a cut-off frequency of the third order Butterworth low-pass filter circuit 44 to about 500 mHz as mentioned previously.

Node 72 feeds into the signal path A output attenuator 84.

The signal path A output attenuator 84 consists of a voltage divider including resistors 902 and 904 to drop the polarized piezoelectric film sensor based signal component to acceptable levels of the PSG machine to which the polarized piezoelectric film sensor is being interfaced via a pair of lead wires 90 and 92 respectively.

The input terminal 22 to the differential amplifier and integrator with resistive reset 60 is coupled, via resistor 602 to the inverting input of operational amplifier 616, to the gain setting and integrator-resetting resistor 610 and to the integrating capacitor 612. The input terminal 24 to the differential amplifier and integrator with resistive reset 60 is coupled, via resistor 604 to the non-inverting input of operational amplifier 616 and to the input load resistor 608.

The output from the differential input amplifier circuit 616 appears at junction 62 and connects to the signal path B third order Butterworth low-pass filter circuit 64.

Referring to filter circuit 64, the input appearing at junction 62 is applied, via series connected resistors 642, 654 and 650, to the non-inverting input of an operational amplifier 660 and those resistors, along with capacitors 646, 652 and 658 cooperate with the operational amplifier 660 to function as a low-pass filter. The output of the operational amplifier 660 is presented to node 74.

The values of the resistors 642, 648 and 650 and the capacitors 646, 654 and 658 may be set to establish a cut-off frequency of the third order Butterworth low-pass filter circuit 64 to about 500 mHz as mentioned previously.

Node 74 feeds into the signal path B output attenuator 88.

The signal path B output attenuator 88 consists of a voltage divider including resistors 962 and 964 to drop the polarized piezoelectric film sensor based signal component to acceptable levels of the PSG machine to which the polarized piezoelectric film sensor is being interfaced via a pair of lead wires 96 and 98 respectively.

Signal nodes 72 and 74 feed, via resistors 802 and 804 respectively into the signal A and signal B summing node 803 of circuit 80. The inverting input of operational amplifier 806 is also the first stage of the inverting integrating integrator with resistive reset circuit 80. The non-inverting input of the operational amplifier 806 connects to virtual ground 59.

Resistor 808 sets the first stage amplifier gain to unity and resets the integrating capacitor 810 which it is connected to in parallel. The integrating capacitor 810 is connected on one side to the summing node 803 and the inverting input of the operational amplifier 806. The other side of the integrating capacitor 810 is connected to the operational amplifier output node 812. Resistor 808 and capacitor 810 set up the averaging RC (resistance times capacitance) time constant for the first integrator stage. The first stage averaging signal integrator 806 is operating with a fixed time constant of around 62.5 ms that has been selected and found to be working optimally during operation and performance regarding respiratory effort.

The output of the first inverting integrator with resistive reset 812 feeds into the inverting input terminal of the second inverting integrator with resistive reset stage via input resistor 814.

The inverting input of operational amplifier 816 is also the second stage of the inverting integrating integrator with resistive reset 80. The non-inverting input of the operational amplifier 816 connects to virtual ground 59.

Resistor 822 sets the second stage amplifier gain to unity and resets the integrating capacitor 820 which it is connected to in parallel. The integrating capacitor 820 is connected on one side to the input resistor 814 and the inverting input of the operational amplifier 816. The other side of the integrating capacitor 820 is connected to the operational amplifier output node 82. Resistor 822 and capacitor 820 set up averaging RC time constant for the second integrator stage. The second stage averaging signal integrator is preferably also operating with a fixed time constant of about 62.5 ms that has been selected and found to be working optimally during operation and performance regarding respiratory effort.

Node 82 feeds into the signal path A+B output attenuator 86.

The signal path A+B output attenuator 86 consists of a voltage divider including resistors 942 and 944 to drop the polarized piezoelectric film based signal component to acceptable levels of the PSG machine to which the polarized piezoelectric film sensor is being interfaced via a pair of lead wires 94 and 95 respectively.

One embodiment with specifically selected components of this invention was found to be operating optimally. The list of specific components used to assemble a printed circuit board assembly is known in the industry as a Bill-of-Materials (BOM). Below is the BOM for one embodiment of this invention we found to be working optimally:

| B1 | BR2330A/FA | |
|---|---|---|
| C1 | 0.1 | uF |
| C2 | 0.1 | uF |
| C3 | 0.1 | uF |
| C4 | 0.1 | uF |
| C5 | 0.39 | uF |
| C6 | 0.056 | uF |
| C7 | 0.1 | uF |
| C8 | 0.1 | uF |
| C9 | 0.39 | uF |
| C10 | 0.056 | uF |
| C11 | 10 | uF/tant |

| | |
|---|---|
| R1 | 1.00M |
| R2 | 100k |
| R3 | 100k |
| R4 | 10.0k |
| R5 | 1.00M |
| R6 | 1.00M |
| R7 | 1.00M |
| R8 | 100k |
| R9 | 1.00k |
| R10 | 100k |
| R11 | 2.70M |
| R12 | 100k |
| R13 | 100k |
| R14 | 1.00M |
| R15 | 100k |
| R16 | 100k |
| R17 | 100k |
| R18 | 10.0k |
| R19 | 1.00M |
| R20 | 1.00M |
| R21 | 1.00M |
| R22 | 1.00M |
| R23 | 1.00k |
| R24 | 100k |
| R25 | 2.70M |
| R26 | 330k |
| R27 | 330k |
| U1: A | LMC6442AIM |
| U1: B | LMC6442AIM |
| U2: A | LMC6442AIM |
| U2: B | LMC6442AIM |
| U3: A | LMC6442AIM |
| U3: B | LMC6442AIM |

During operation in a typical application, such as in a sleep laboratory, a patient is fitted with a belt-mounted polarized piezoelectric film sensor, that includes the circuit that has been described in detail here in order for sleep scientists, sleep physicians and sleep technicians to see, detect and properly diagnose specific sleep disorders and diseases which including abnormal respiratory events including events occurring in the upper airway of the patient.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

The description of the various embodiments is merely exemplary in nature and, thus, variations that do not depart from the gist of the examples and detailed description herein are intended to be within the scope of the present disclosure. Such variations are not to be regarded as a departure from the spirit and scope of the present disclosure.

What is claimed is:

1. An apparatus, comprising:
an electronic signal processing circuit configured to receive first information indicative of respiratory effort of a subject from a first piezoelectric film sensor and second information indicative of respiratory effort of the subject from a second piezoelectric film sensor, wherein the electronic signal processing circuit is configured to process the received first and second information to produce an electronic signal output indicative of respiratory effort of the subject, the electronic signal processing circuit including:
a first differential amplifier and signal integrator with resistive reset configured to average the received first information to reduce differential noise and to attenuate common-mode noise; and
a second differential amplifier and signal integrator with resistive reset configured to average the received second information to reduce differential noise and to attenuate common-mode noise.

2. The apparatus of claim 1, including:
a first respiratory effort belt including the first piezoelectric film sensor configured to sense movement indicative of respiratory effort of the subject; and
a second respiratory effort belt including the second piezoelectric film sensor configured to sense movement indicative of respiratory effort of the subject.

3. The apparatus of claim 2, wherein the first respiratory effort belt includes a chest movement respiratory effort belt and the second respiratory effort belt includes an abdominal movement respiratory effort belt.

4. The apparatus of claim 2, wherein the first and second piezoelectric film sensors include independent polarized piezoelectric film sensors configured to provide a rigid phase and polarity relationship between respiratory effort movement.

5. The apparatus of claim 1, wherein the first and second differential amplifiers and signal integrators with resistive resets are configured to provide a predetermined gain factor by which the received first and second information is amplified, to provide signal averaging over time to reduce differential noise, and to attenuate common-mode noise.

6. The apparatus of claim 1, wherein the electronic signal processing circuit includes:
a first third order Butterworth low pass filters configured to remove components from the received first information having a frequency above approximately 500 mHz; and
a second third order Butterworth low pass filters configured to remove components from the received second information having a frequency above approximately 500 mHz.

7. The apparatus of claim 1, wherein the first and second differential amplifiers and signal integrators with resistive reset are configured to average the received first and second information only during the subject's respiratory response time.

8. The apparatus of claim 1, wherein the electronic signal output indicative of respiratory effort of the subject includes a summation of the averaged received first and second information.

9. The apparatus of claim 1, wherein the electronic signal output indicative of respiratory effort of the subject includes the averaged received first information, and separately, the averaged received second information.

10. The apparatus of claim 1, wherein the electronic signal output indicative of respiratory effort of the subject includes, separately, the averaged received first information, the averaged received second information, and a summation of the averaged received first and second information.

11. A system, comprising:
a first respiratory effort belt including a first piezoelectric film sensor configured to sense a first movement indicative of respiratory effort of a subject;
a second respiratory effort belt including a second piezoelectric film sensor configured to sense a second movement indicative of respiratory effort of the subject;
an electronic signal processing circuit, coupled to the first and second piezoelectric film sensors, the electronic signal processing circuit configured to receive first and second information from the first and second piezoelectric film sensors and to process the received first and second information to produce an electronic signal output indicative of respiratory effort of the subject;
wherein the electronic signal processing circuit includes:
a first differential amplifier and signal integrator with resistive reset configured to average the received first information to reduce differential noise and to attenuate common-mode noise; and
a second differential amplifier and signal integrator with resistive reset configured to average the received second information to reduce differential noise and to attenuate common-mode noise in the second;
wherein the electronic signal output indicative of respiratory effort of the subject includes:
the averaged received first information;
the averaged received second information; and
a summation of the averaged received first information and the averaged received second information; and
a polysomnograph machine, coupled to the electronic signal processing circuit, the polysomnograph machine configured to receive the averaged received first information, the averaged received second information, and the summation of the averaged received first information and the averaged received second information from the electronic signal processing circuit and to provide the averaged received first information, the averaged received second information, and the summation of the averaged received first information and the averaged received second information to a user.

12. A method, comprising:
receiving first information indicative of respiratory effort of a subject from a first piezoelectric film sensor and second information indicative of respiratory effort of the subject from a second piezoelectric film sensor; and
processing the received first and second information to produce an electronic signal output indicative of respiratory effort of the subject, the processing including:
averaging the received first information using a first differential amplifier and signal integrator with resistive reset to reduce differential noise and to attenuate common-mode noise; and
averaging the received second information using a second differential amplifier and signal integrator with resistive reset to reduce differential noise and to attenuate common-mode noise.

13. The method of claim 12, wherein the receiving the first and second information includes receiving first and second information from the first and second piezoelectric film sensors coupled to respective first and second respiratory effort belts.

14. The method of claim 13, wherein the first respiratory effort belt includes a chest movement respiratory effort belt and the second respiratory effort belt includes an abdominal movement respiratory effort belt.

15. The method of claim 13, wherein the first and second piezoelectric film sensors includes independent polarized piezoelectric film sensors; and
wherein the receiving the first and second information includes receiving rigid phase and polarity relationship information between respiratory effort movement from the independent polarized piezoelectric film sensors.

16. The method of claim 12, wherein the processing includes amplifying the received first and second information by a predetermined gain factor using the first and second differential amplifiers and signal integrators with resistive reset, to provide signal averaging over time to reduce differential noise, and to attenuate common-mode noise.

17. The method of claim 12, wherein the processing includes removing components from the received first and second information having a frequency above approximately 500 mHz using respective first and second third order Butterworth low pass filters.

18. The method of claim 12, wherein the averaging the received first and second information includes averaging only during the subject's respiratory response time.

19. The method of claim 12, wherein the producing the electronic signal output indicative of respiratory effort of the subject includes producing a summation of the averaged received first and second information.

20. The method of claim 19, wherein the producing the electronic signal output indicative of respiratory effort of the subject includes producing the averaged received first information, and separately, the averaged received second information.
